(19) United States
(12) Patent Application Publication
(10) Pub. No.: US 2017/0326213 A1
(43) Pub. Date: Nov. 16, 2017

(54) Protein-Coupled Red Blood Cell Compositions and Methods of Their Use

(71) Applicant: Augusta University Research Institute, Inc., Augusta, GA (US)

(72) Inventors: Ryan Jajosky, North Augusta, SC (US); Rafal Pacholczyk, Martinez, GA (US); Gurmukh Singh, Augusta, GA (US)

(21) Appl. No.: 15/596,586

(22) Filed: May 16, 2017

Related U.S. Application Data
(60) Provisional application No. 62/336,896, filed on May 16, 2016.

(51) Int. Cl. A61K 39/00; A61K 9/19

(52) U.S. Cl. CPC A61K 39/0008; A61K 9/19; A61K 2039/6006; A61K 2039/515; A61K 2039/627; A61K 2039/577

(57) ABSTRACT

Methods and compositions for using N-hydroxysuccinimide and N-hydroxysulfosuccinimide to covalently couple protein(s) to the surface of red blood cells universally, rapidly and conveniently are provided. In one embodiment, the compositions promote immune tolerance in a subject. One embodiment provides autologous or allogenic red blood cells having whole protein(s) of interests conjugated to proteins on or within the plasma membrane of the red blood cells, wherein the conjugated proteins display at least one antigen to which immune tolerance is desired. The proteins are conjugated to the RBCs using carbodiimide chemistry. In a preferred embodiment, the whole proteins are conjugated using EDC in combination with NHS or sulfo-NHS.

### FIELD OF THE INVENTION

One aspect of the invention is generally directed to peptide/protein coupled red blood cell (RBC) compositions and methods of their use.

### BACKGROUND OF THE INVENTION

Antigen-specific immune tolerance is a highly desired therapy for immune-mediated diseases (Getts, D. R., et al., J. Immunol., 187(5):2405-2417 (2011)). Most existing therapies for treating autoimmune disease involve general immunosuppression, which involves suppressing the body's entire immune system. General immunosuppression is associated with severe life-threatening adverse events (e.g., infection, cancer, etc.). Antigen-specific immune tolerance aims to suppress only the specific unwanted immune attack (e.g., the immune attack against the autoantigen in the body). Antigen-specific immune tolerance is desirable because it is more effective and safer than general immunosuppression (not associated with infection, cancer, etc.).

Intravenous infusion of peptide/protein antigens linked to leukocytes (blood mononuclear cells, lymphocytes, etc.) with 1-ethyl-3-(3-dimethylaminopropyl)carbodiimide hydrochloride (EDC) is a highly efficient method for inducing peripheral, antigen-specific T cell tolerance for the treatment of autoimmune disease. Lutterotti, A. et al. disclose using EDC to couple myelin peptides to autologous peripheral blood mononuclear cells (PBMCs) for treating multiple sclerosis (Lutterotti, A., et al., Sci. Transl. Med. 5, 188ra77 (2013)). Leukocytes can be easily collected from mice, by harvesting the spleen. However, in human patients, leukocytes are collected using leukapheresis, a procedure which is time-consuming, expensive, and associated with severe complications.

Red blood cells (RBCs) are also being used as carriers for intravascular drug delivery, characterized by unique longevity in the bloodstream (approximately 120-day lifespan for healthy adult human RBCs), biocompatibility, and safe physiologic mechanisms for metabolism (splenic and hepatic macrophages and antigen-presenting cells). Thus, RBCs are being explored as an advanced-drug-delivery vehicle (Villa, C. H., et al., Adv Drug Deliver Rev. 106(Pt A):88-103 (2016) for delivering drugs and other biologically active substances (V. Muzykantov, Expert Opin Drug Deliv. 7(4):403-427 (2010)). Drugs can either be encapsulated within the RBCs, or attached to the surface of the RBCs.

Internal loading of proteins into RBCs is being extensively researched (Hunault-Berger, M., et al., Am J Hematol. 90(9):811-8 (2015)). Internal loading is achieved using hypo-osmotic/hypotonic stress, which opens pores in the RBC membrane. After loading the pores are closed by normalizing the osmotic pressure. However internal loading methods are expensive because large-scale manufacturing requires the use of dialysis machinery which is located at a centralized manufacturing site (Domenech, C., et al. Br J Haematol. 153(1):58-65 (2011)). In addition, because of centralized manufacturing at a cGMP pharmaceutical unit, allogenic (homologous) rather than autologous RBCs are used (Domenech, C., et al. Br J Haematol. 153(1):58-65 (2011)). Allogenic RBCs are associated with adverse events such as infectious diseases (e.g. HIV virus, HBV virus, HCV virus, etc.) and non-infectious diseases (e.g. transfusion related acute lung injury or TRALI, transfusion associated graft-versus-host disease or TA-GVHD, anaphylaxis, etc.).

Loading peptides/proteins onto the surface of RBCs has not been fully explored. RBCs which are genetically engineered have be linked to residues 35-55 of myelin oligodendrocyte glycoprotein (MOG₃₅₋₅₅) and residues 9-23 of insulin (Ins₉₋₂₃). Transfusion of RBCs carrying MOG₃₅₋₅₅ can prevent and reverse signs of multiple sclerosis in mice (Pishesha, N., et al., Proc Natl Acad Sci USA. 114(12):3157-3162 (2017)). Transfusion of RBCs carrying Ins₉₋₂₃ confer protection against type 1 diabetes (Pishesha, N., et al., Proc Natl Acad Sci USA. 114(12):3157-3162 (2017)). However, production of genetically engineered RBCs is labor-intensive, expensive, and takes weeks-months.

Alternatively, RBCs can be non-covalently linked to proteins using antibodies or fragments of antibodies. The Ter¹¹⁹ antibody binds to rat/mouse glycophorin A protein, which is present on rat and mouse RBCs. A single chain variable fragment of antibody Ter¹¹⁹, which is the portion of the antibody which binds to glycophorin A (Ter¹¹⁹ scFv) was then fused with thrombomodulin (TM) to create scFv/TM. The scFv/TM fusion protein can then be administered intravenously, so that it will bind to glycophorin A on RBCs in the bloodstream, to improve the pharmacokinetics and antithrombotic effects of thrombomodulin (Carnemolla, R., et al., FASEB J. 31(2):761-770 (2017)). Alternatively, a protein can be non-covalently linked to RBCs by fusing the protein-of-interest with a peptide which has a high affinity for a specific RBC antigen. The ERY1 peptide was designed to have a high affinity for mouse glycophorin A, which is found on mouse RBCs (Kontos, S., et al. Proc Natl Acad Sci USA. 110(1):E60-8 (2013)). However, non-covalently linking proteins to RBCs with antibodies/peptides only works if a specific antigen, such as glycophorin A, is present on the surface of the RBC. Patient RBCs may not have glycophorin A (MkMk phenotype) or the RBCs may have a variant of glycophorin A due to a mutation (e.g. single nucleotide polymorphism). Additionally, the ERY1 peptide does not bind to human RBCs. In either case, the non-covalent linking methods using antibodies or peptides will not work for all patient RBC samples.

An alternative method exploits the non-covalent strong binding affinity of biotin and streptavidin to link proteins to RBCs (Armstead, W. M, et al. J Cereb Blood Flow Metab. 29(8):1463-1474. (2009)). This involves biotinylation of the RBCs and the peptide/protein of interest. Then, the biotin-RBCs are washed and reacted with streptavidin. The streptavidin forms a non-covalent bond with biotin. Then, the streptavidin-biotin-RBCs are washed and reacted with the biotinylated protein. This yields the following construct “peptide/protein of interest”-biotin-streptavidin-biotin-RBC. However, this method is neither rapid nor convenient. This is because there are three major steps in this reaction with the need for RBC washing between each step.

Covalently linking proteins onto the surface of RBCs, using a chemical reaction, could overcome the challenges associated with surface loading of proteins onto RBCs. However, methods of covalently linking peptides/proteins to the surface of RBCs, are either damaging to the RBCs, labor-intensive, or time-consuming. V. Muzykantov discloses that using cross-linking agents and procedures (e.g. EDC alone) to couple proteins or peptides to RBCs grossly damages the RBC cell membrane, reducing RBC plasticity, resistance to lytic agents and biocompatibility (V. Muzykantov, Expert Opin Drug Deliv. 7(4):403-427 (2010)). In addition, S. Prasad discloses that coupling bovine insulin to RBCs takes 1 hour, which is a significant amount of time, and that the transfusion of bovine insulin linked to RBCs (made using EDC) results in “cell lysis, release of hemoglobin and toxicity” (Prasad, S., et al. J Autoimmun. 39(4):347-353 (2012)). Thus, EDC crosslinking is a time-intensive process, damaging to the RBCs, and results in unwanted toxicity and hemolysis.

Other methods of covalently linking proteins onto the surface of RBCs require “linker compounds”, which remain as a physical bridge between the RBC and the protein of interest. This can be written as follows: RBC-“linker compound”—“protein-of-interest”. For example, “sortagging” can be used to link proteins onto RBCs. However, “sortagging” produces a final product with a “linker compound” within it (Pishesha, N., et al. Proc Natl Acad Sci USA. 114(12):3157-3162 (2017)). The crosslinker sulfosuccinimidyl 4-(N-maleimidomethyl)cyclohexane-1-carboxylate (also known as sulfo-SMCC) has been used to attach proteins onto RBCs. However, a “linker compound” is also present in the final product. Additionally, the quantity of protein attached to the RBC is low because sulfo-SMCC activated proteins react with free sulfhydryl groups, which are uncommon on the surface of RBCs (Kontos, S., et al. Proc Natl Acad Sci USA. 110(1):E60-8 (2013)). The crosslinker LC-SPDP has also been used to attach proteins onto RBCs. However, a “linker compound” is also present in the final product (Zimring, J. C., et al. Blood. 107(1):187-9 (2006)). The LC-SPDP activated protein is reactive toward free sulfhydryl groups on the RBCs. However, because there are so few free sulfhydryl groups on RBCs, the RBCs had to be reacted with 2-iminothiolane (Traut's reagent) for 30 minutes prior to the reaction. The LC-SPDP-modified protein then had to be reacted with the Traut reagent-modified RBCs for 2 hours, which is time-consuming. Having a “linker compound” act as a structural bridge between the protein-of-interest and the RBC is undesirable, because the safety profile of the “linker compound” is unknown (potentially toxic) and is often immunogenic (Hermanson, G T. Bioconjugate Techniques. Academic Press (2013)).

The approach of using cell transplantation to achieve immune tolerance continues to be pioneered by Dr. Stephen D. Miller (Northwestern University). In his career, which has spanned multiple decades, he used EDC (sometimes abbreviated ECDI) to link peptides/proteins onto cellular carriers (Xunrong, L., et al. Annu Rev Biomed Eng. 18:181-205 (2016)). Dr. Miller is the clear expert and leader in this field. However, Dr. Miller never used NHS/sulfo-NHS compounds to link peptides/proteins onto cells. In addition, Dr. Miller never used NHS/sulfo-NHS compounds to link peptides/protein onto RBCs. In addition, no individual has done such, and no individual has done such for the purposes stated above.

Thus, there is a need for improved methods by which peptides/proteins can be covalently linked to the surface of RBCs for drug delivery, extension of drug/peptide/protein half-life, immune tolerance induction, reduction and elimination of titers of specific antibodies, and immunization. Specifically, there is a need for a method which covalently links peptides/proteins to RBCs which works for all RBCs (universal applicability) and is safe, convenient (not labor-intensive or time-intensive), and inexpensive.

Therefore, it is an object of the invention to provide peptide/protein linked RBC compositions for delivering peptide/protein to macrophages/antigen-presenting cells of the spleen, liver, or bone marrow to either achieve antigen-specific immune tolerance or induce antigen-specific immunization.

It is another object of the invention to provide compositions to deliver peptide/protein linked RBCs to macrophages/antigen-presenting-cells which need specific peptides/proteins.

It is another object of the invention to provide peptide/protein linked RBC compositions to adsorb pathologic antibodies onto the peptide/protein linked RBCs for reducing/eliminating antibody titers.

It is another object of the invention to provide methods by which the half-life of a peptide/protein can be controlled (increased/decreased) by attachment to the RBC and then regulating the amount of damage (e.g. heat damage) inflicted on the peptide/protein linked RBCs.

It is another object of the invention to provide compositions, such as lyophilized pre-weighed quantities of NHS and sulfo-NHS activated peptides/proteins that can be rapidly and conveniently used to covalently link peptides/proteins to the surface of RBCs.

It is still another object of the invention to provide kits for performing the coupling of proteins to red blood cells.

It is still another object of the invention to provide methods for inducing immune tolerance, methods for immunization, methods for antibody adsorption and elimination, and methods for regulating peptide/protein half-life in a subject.

It is still another object of the invention to provide kits for inducing immune tolerance in a subject.

### SUMMARY OF THE INVENTION

Methods and compositions for covalently coupling peptides/proteins to the surface of RBCs are provided. One embodiment provides autologous or allogeneic RBCs having peptides/proteins coupled to native RBC cell membrane proteins. The peptide/protein linked RBCs are transfused (administered intravenously) to the patient as a therapy. Autologous RBCs are preferred to allogeneic, as they are associated with fewer adverse events. The peptides/proteins to be attached to the RBCs can vary in size and structure. The peptides/proteins of interest are “activated” with either N-hydroxysuccinimide (NHS) or N-hydroxysulfosuccinimide (sulfo-NHS). In general, NHS activation is used when the peptide/protein is hydrophobic or highly soluble in organic solvents. Sulfo-NHS activation is used when the peptide/protein is hydrophilic or highly soluble in water or other polar solvents. The NHS/sulfo-NHS activated peptide/protein will then form a covalent bond to mostly primary amine groups found on the surface of the RBC.

Peptide/protein activation with NHS or sulfo-NHS can be achieved by many different methods. For most hydrophilic (water-soluble) peptides/proteins, sulfo-NHS can be used with carbodiimide chemistry with 1-ethyl-3(3-dimethylaminopropyl)carbodiimide (EDC) or 1-cyclohexyl-3-2(2-morpholinoethyl) carbodiimide (CMC) to create an activated peptide/protein. (Carbodiimide chemistry can be used to activate the peptides/proteins, however, it is not required, as TSTU (N,N,N′,N′-Tetramethyl-O-(N-succinimidyl)uronium tetrafluoroborate) can be used.) The reaction of peptide/protein with EDC and sulfo-NHS can occur in a polar solvent, such as phosphate buffered saline (PBS). For most hydrophobic (organic solvent soluble) peptides/proteins, NHS can be used with carbodiimide chemistry with dicyclohexylcarbodiimde (DCC) or diisopropyl carbodiimide (DIC) to create an activated peptide/protein. This reaction would occur in an organic solvent (e.g. dimethyl sulfoxide or DMSO, dimethylformamide or DMF, acetone, dioxane, etc.). In addition, the peptide/protein of interest can be treated with an amine-blocker prior to activation with NHS/sulfo-NHS to prevent aggregation and precipitation.

Another embodiment provides a method for promoting immune tolerance to an antigen in a subject in need thereof by administering allogeneic or autologous RBCs to the subject intravenously, wherein the RBCs have peptide/protein conjugated to the RBCs, and wherein the peptide/protein is the antigen target of the condition or is capable of inducing immune tolerance. The peptide/protein can be autologous or heterologous. Senescent/damaged RBCs are recycled by tolerance-inducing macrophages/antigen-presenting cells of the spleen, liver, and bone marrow. Therefore, the RBC conjugated peptides/proteins are delivered and targeted to these immune-tolerance-inducing cells.

Alternatively, another embodiment provides a method for promoting immunization to a peptide/protein in a subject in need thereof by administering allogeneic or autologous RBCs to the subject, wherein the RBCs are conjugated to peptides/proteins, and wherein the peptide/protein is the antigen to be immunized against. A preferred subject is human.

Another embodiment provides methods by which peptide/protein coupled RBCs adsorb and eliminate pathologic antibodies in patients, directed against or cross-reactive with the peptide/protein antigen coupled to the RBCs. Antibodies bind to the peptide/protein on the RBCs and the antibody-coated-RBCs are recycled by macrophages and antigen-presenting cells. Thus, the antibody will be removed from the circulation.

Another embodiment provides a method for controlling (increasing or decreasing) the half-life of the peptide/protein, which is coupled to the RBC, depending on the desired outcome. Many soluble peptides are cleared from the circulation within minutes to hours of infusion. Because human adult RBCs have a lifespan of about 120 days, RBCs can extend the half-life of the peptide/protein. Conversely, damaged RBCs (heat damaged or chemically damaged) are cleared from the circulation by macrophages/antigen presenting cells faster than healthy RBCs. Regulating the extent/severity of damage can be used to regulate the half-life of the peptide/protein (carried on the RBCs). The peptide/protein on damaged RBCs can be rapidly delivered (short half-life) to macrophages/antigen-presenting cells of the spleen, liver, and bone marrow. Representative proteins to be conjugated to RBCs include, but are not limited to insulin, proinsulin, preproinsulin, glutamic acid decarboxylase-65 (GAD-65), thyroglobulin (TG), thyroid peroxidase (TPO), thyrotropin receptor (TSHR), collagen II, islet antigen-2 (IA-2), myelin oligodendrocyte glycoprotein (MOG), myelin basic protein (MBP), myelin proteolipid protein (PLP), coagulation factor VIII, factor IX, factor X, factor XIII, factor V, thrombin, RBC antigens (Rh, including D, C, c E, and e antigens, Kidd, Kell, etc), class I and class II HLA molecules, asparaginase, CD55, CD59, gluten, erythropoietin, galactosidase, glucuronidase, glucocerebrosidase, acid alpha-glucosidase, adenosine deaminase, tissue plasminogen activator, β1-adrenergic receptor, allergens (peanut allergens), microbial antigens, anti-cancer agents, tumor antigens, U1snRNP, SS-A, high mobility group box 1 (HMGB1), nucleosomes, histone proteins, nuclear proteins, glomerular basement membrane proteins including collagen IV, cardiac myosin, tissue transgluaminase, gliadin, tyrosinase, tyrosinase related protein 1 and 2, desmoglein 3, 1 and 4, BP180 and BP230 reticulin, 21-hydroxylase, 17-hydroxylase, collagen VII, the cytochrome P450 side chain cleavage enzyme, gastric H+/K+-ATPase, intrinsic factor, Fc portion of IgG, topoisomerase I, ribosomal P, alpha-subunit of the nicotinic acetylcholine receptor, muscle-specific kinase, low-density lipoprotein receptor-related protein 4, voltage-gated potassium channel, topoisomerase I, ribosomal P, aquaporin-4, N-methyl-D-aspartate receptor, and tRNA synthetase. Yet another embodiment provides a kit containing an aliquot(s) of amine-blocked or non-amine-blocked peptide/protein activated by NHS or sulfo-NHS to form NHS or sulfo-NHS esters of the carboxylic acids of the peptide/protein, a separate non-amine containing buffer solution pH 7.2-7.6, with or without a cell washing device or equipment based on microfluidics, with or without reagents/testing device for confirming the attachment of peptide/protein on the RBC, and instructions for conjugating the peptide/protein to RBCs obtained from a subject. Exemplary carbodiimides used in the activation process are 1-ethyl-3-(3-dimethylaminopropyl)carbodiimide (EDC) and dicyclohexylcarbodiimde (DCC). Preferred agents are N-hydroxysuccinimide (NHS) and N-hydroxysulfosuccinimide (sulfo-NHS).

### BRIEF DESCRIPTION OF THE DRAWINGS

FIGS. 1A-1C are schematic diagrams of an exemplary process for coupling peptides/proteins to RBCs.

FIGS. 2A-2D are schematic diagrams showing different methods used to crosslink OVA to RBCs. FIG. 2A shows EDC inefficiently crosslinks OVA to RBCs. FIG. 2B shows Sulfo-NHS-OVA effectively crosslinks OVA to RBCs. FIG. 2C shows sulfo-NSH-b-OVA efficiently crosslinks OVA to RBCs.

FIGS. 3A-1 to 3A-6 are flow cytometry dot plots. FIG. 3B is a bar graph of “SSC” tail percentage for the identified coupling method. FIGS. 3C-1 to 3C-6 are histograms showing RBCs reacted with suflo-NHS-n-OVA or sulfo-NHS-b-OVA had an average of more than 1000 OVA molecules attached per RBC. FIG. 3D is a graph of OVA per RBC versus time (minutes). N-RBCs=normal RBCs, n-OVA=normal OVA, b-OVA=amine blocked OVA

FIGS. 4A-4J are flow cytometry dot plots showing transfused N-RBCs and OCA-RBCs in circulation.

FIG. 5 is a line graph of transfused RBCs in circulation versus post-transfusion time (weeks).

FIGS. 6A-6C are flow cytometry dot plots of N-RBCs, OVA-RBCs without antibody, and OVA-RBCs with antibody, respectively.

FIG. 7A is a graph of days in storage for the RBCs of the indicated blood type. FIG. 7B is a graph of OVA molecules per RBC.

FIG. 8 is a line graph of percentage of total OVA-RBC remaining in circulation versus time (hours). Heat damaged (48.50 ° C.) ovalbumin-coupled mouse RBCs are cleared from the circulation faster than undamaged (room temperature) RBCs. The heat damage was at 48.5° C. for 20 minutes.

FIG. 9 is a graph of adjusted MFI for indicated RBCs. p <0.05.

FIG. 10 is a graph of adjusted MFI for indicated RBCs. Mice received a first transfusion (2×10̂8 RBCs), then 6 weeks later received a second transfusion (2×10̂8 RBCs). Six weeks after the second transfusion, the anti-OVA IgG titers were measured using a flow cytometric crossmatch. Mice receiving OVA(lo)-RBCs followed by OVA(hi)-RBCs had anti-OVA IgG titers that were not significantly different than mice that only received RBCs. Conversely, OVA(hi)-RBCs followed by OVA(hi)-RBCs had anti-OVA IgG titers which were significantly higher than mice receiving OVA(lo)-RBCs followed by OVA(high)-RBCs and mice receiving RBCs followed by RBCs. p <0.05

FIG. 11 is a graph of percentage of activated OVA-specific CD8+ T cells for ova immunization, OVA(lo)-RBCs and innumization and RBCs. Mice either received OVA immunization only, OVA(lo)-RBCs followed by OVA immunization (Poly I:C) 2 weeks later, or RBCs only. The spleens were then harvested (4 weeks post-immunization or post-RBC transfusion), and the OVA-specific CD8+ CD3+ T cells were selected. The percentage of CD62L negative cells, which represents the activated CD8 T cells, was then measured. Mice receiving OVA(lo)-RBCs prior to immunization with OVA had significantly lower percentages of activated OVA-specific CD8+ T cells compared with mice only immunized against OVA. The percentages of OVA-specific CD8+ T cells was not significantly different for mice receiving OVA(lo)-RBCs prior to immunization and mice receiving only RBCs. p <0.05.

FIG. 12 is a bar graph of adjusted MFI for RBC treatment or OVA-RBC treatment. Mice were immunized against OVA using Poly I:C and developed anti-OVA IgG antibodies. Then, mice were treated with either 5×10̂8 of RBCs or OVA-RBCs. The OVA-RBCs had ˜1100 OVA molecules per RBC. The OVA-RBC treated group had significantly reduced antibody titers 2 hours after treatment, while the RBC treated group did not. p <0.05.

### DETAILED DESCRIPTION OF THE INVENTION

### I. Definitions

The use of the terms “a,” “an,” “the,” and similar referents in the context of describing the presently claimed invention (especially in the context of the claims) are to be construed to cover both the singular and the plural, unless otherwise indicated herein or clearly contradicted by context.

Recitation of ranges of values herein are merely intended to serve as a shorthand method of referring individually to each separate value falling within the range, unless otherwise indicated herein, and each separate value is incorporated into the specification as if it were individually recited herein.

Use of the term “about” is intended to describe values either above or below the stated value in a range of approx. +/−10%; in other embodiments the values may range in value either above or below the stated value in a range of approx. +/−5%; in other embodiments the values may range in value either above or below the stated value in a range of approx. +/−2%; in other embodiments the values may range in value either above or below the stated value in a range of approx. +/−1%. The preceding ranges are intended to be made clear by context, and no further limitation is implied. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

The term “antigen” refers to a substance that is capable of stimulating an immune response. In general, two main divisions of antigens are recognized: foreign antigens (or heteroantigens) and autoantigens (or self-antigens). Foreign antigens originate from outside the body. An antigen can be a protein, peptide, polysaccharide, DNA, RNA, or small molecules coupled to carrier proteins (haptens). An autoantigen refers to an antigen that is a normal bodily constituent and against which the immune system produces autoantibodies.

The term “APEX” stands for activated-protein erythrocyte crosslinking.

The term “autoantibody” refers to an antibody produced by a subject's immune system that is directed against one or more of the subject's own proteins.

The term “immune tolerance” refers to a state of unresponsiveness to a specific antigen or group of antigens to which a subject is normally responsive. Immune tolerance is achieved under conditions that suppress the immune reaction and is not just the absence of an immune response.

The term “RBC” refers to Red Blood Cell.

The term “EDC” refers to 1-ethyl-3-(3-dimethylaminopropyl)carbodiimide hydrochloride

The term “NHS” refers to N-hydroxysuccinimide

The term “Sulfo-NHS” refers to N-hydroxysulfosuccinimide.

The term “subject” refers to an animal, preferably a human.

### II. Protein-Coupled Red Blood Cell Compositions

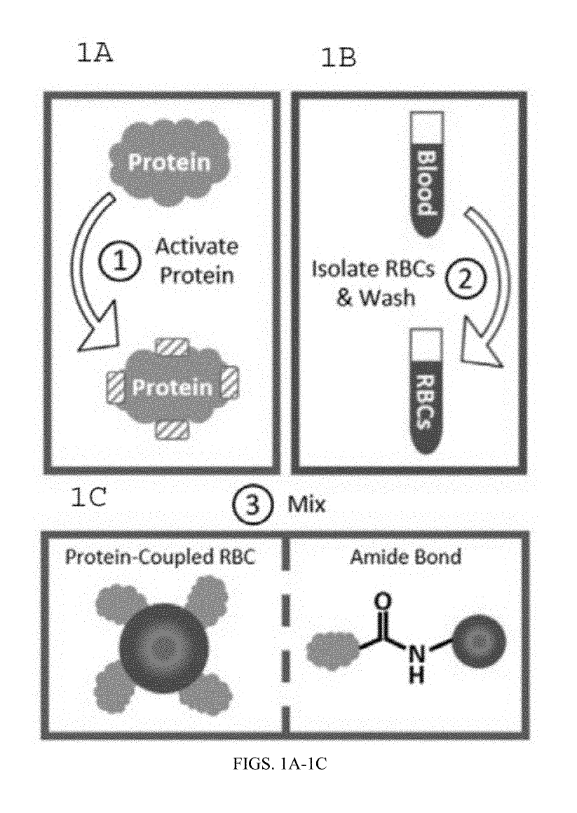

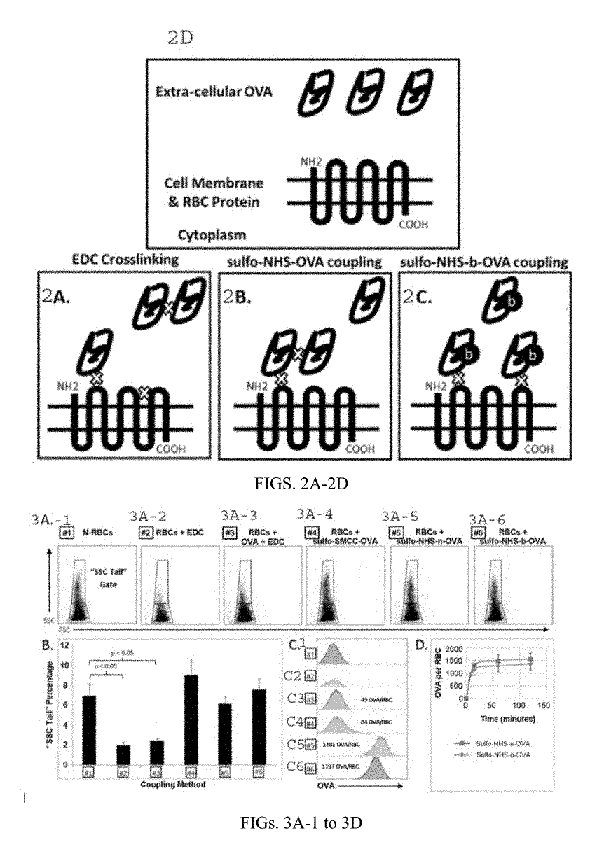

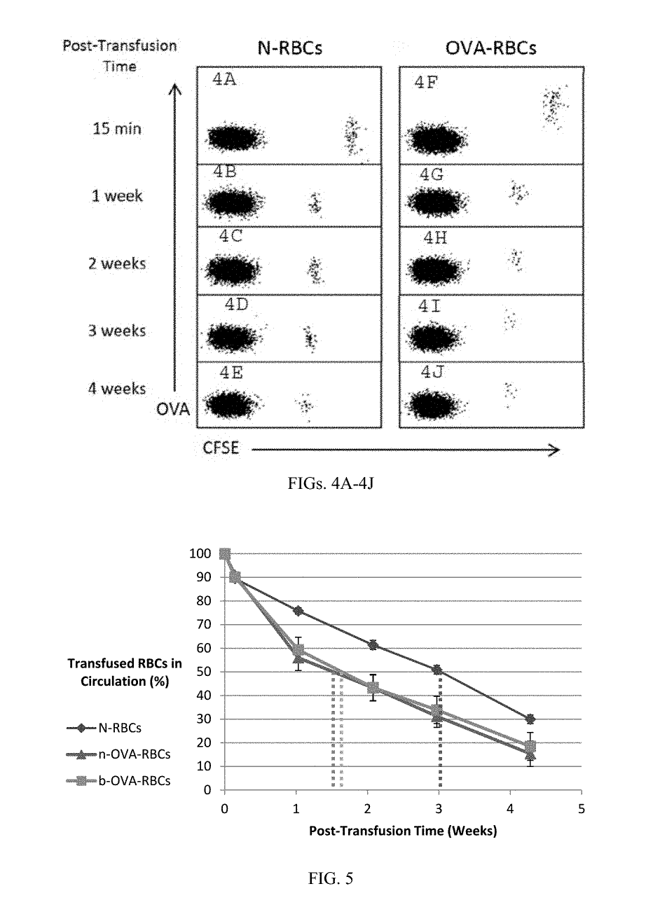

It has been discovered that peptides/proteins can be conjugated to primary amine groups found on RBC-membrane proteins without causing damage/hemolysis of the RBCs by using NHS or sulfo-NHS activated peptides/proteins. Preferably, the coupling of peptides/proteins to RBCs is done in a closed system using good manufacturing practice (GMP). NHS or sulfo-NHS esters principally cross-link principally to alpha-amines on N-terminal amino acids and epsilon-amines of lysine side chains. FIG. 1. depicts a diagram of how NHS/sulfo-NHS activated peptides/proteins attach to RBCs. FIG. 2 depicts how NHS/sulfo-NHS activated peptides/proteins do not damage RBCs, because cross-linking of native RBC proteins to native RBC proteins does not occur, as it does with EDC. FIG. 3A/B. depicts how EDC crosslinking ovalbumin protein (OVA) onto RBCs damages the RBCs causing the RBC to loss Side Scatter (SS), while RBCs treated with sulfo-NHS activated OVA and sulfo-NHS-activated “amine blocked OVA” are not damaged. Sulfo-NHS-n-OVA is normal ovalbumin activated by sulfo-NHS, while Sulfo-NHS-b-OVA is amine blocked ovalbumin activated by sulfo-NHS. FIG. 3C. depicts how sulfo-NHS-n-OVA and sulfo-NHS-b-OVA attach significantly more OVA onto RBCs than EDC. FIG. 3D. depicts how rapidly sulfo-NHS-n-OVA and sulfo-NHS-b-OVA attach OVA onto RBCs. More than 80% of the OVA attached to the RBC occurs in the first 15 minutes of the reaction. The reaction can be stopped quickly or allowed to proceed over a longer time period to decrease or increase the amount of peptide/protein attached to the RBCs. FIG. 4/5. Depict the circulation of OVA-RBCs made using sulfo-NHS-n-OVA and sulfo-NHS-b-OVA. The OVA attached to these RBCs is still detectable after 4 weeks in circulation, in mice.

In a preferred embodiment, DCC and NHS or EDC and sulfo-NHS are used together to create a NHS or sulfo-NHS activated peptide/protein. The peptide/protein is preserved through the cross-linking reaction and can be used to induce tolerance to the antigens when the protein-conjugated RBCs are administered to a subject. The antigens of the peptide/protein may or may not have their three-dimensional shape altered, depending on the desired outcome. A peptide/protein with a properly folded three-dimensional shape is likely to be needed for antibody adsorption and elimination. However, a peptide/protein needed for immune tolerance induction may not need to be properly folded in order to achieve immune tolerance.

If a peptide/protein without its native three-dimensional conformation is desired, then there are several options to achieve this. A misfolded peptide/protein can be synthesized using organic chemistry. Otherwise using a recombinant peptide/protein expression system such as bacteria, yeast, plants, or insects can yield a misfolded peptide/protein. Otherwise well-described denaturation protocols can be performed using hot/cold temperatures, acids, bases, solvents (e.g. ethanol, alcohol, etc.), cross-linking agents (e.g. formaldehyde, etc.), chaotropic agents (e.g. urea 6-8 mol/l, etc.), or disulfide bond reducers (e.g. 2-mercaptoehtanol, dithiothreitol, etc.).

A. Proteins Conjugated to RBCs

The peptide/protein can be conjugated to RBCs using NHS/sulfo-NHS activated peptides/proteins. Representative proteins to be conjugated to RBCs include, but are not limited to insulin, proinsulin, preproinsulin, glutamic acid decarboxylase-65 (GAD-65), thyroglobulin (TG), thyroid peroxidase (TPO), thyrotropin receptor (TSHR), collagen II, islet antigen-2 (IA-2), myelin oligodendrocyte glycoprotein (MOG), myelin basic protein (MBP), myelin proteolipid protein (PLP), coagulation factor VIII, factor IX, factor X, factor XIII, factor V, thrombin, RBC antigens (Rh, including D, C, c E, and e antigens, Kidd, Kell, etc), class I and class II HLA molecules, asparaginase, CD55, CD59, gluten, erythropoietin, galactosidase, glucuronidase, glucocerebrosidase, acid alpha-glucosidase, adenosine deaminase, tissue plasminogen activator, β1-adrenergic receptor, allergens (peanut allergens), microbial antigens, anti-cancer agents, tumor antigens, U1snRNP, SS-A, high mobility group box 1 (HMGB1), nucleosomes, histone proteins, nuclear proteins, glomerular basement membrane proteins including collagen IV, cardiac myosin, tissue transgluaminase, gliadin, tyrosinase, tyrosinase related protein 1 and 2, desmoglein 3, 1 and 4, BP180 and BP230 reticulin, 21-hydroxylase, 17-hydroxylase, collagen VII, the cytochrome P450 side chain cleavage enzyme, gastric H+/K+-ATPase, intrinsic factor, Fc portion of IgG, topoisomerase I, ribosomal P, alpha-subunit of the nicotinic acetylcholine receptor, muscle-specific kinase, low-density lipoprotein receptor-related protein 4, voltage-gated potassium channel, topoisomerase I, ribosomal P, aquaporin-4, N-methyl-D-aspartate receptor, and tRNA synthetase.

Any peptide/protein to which immune tolerance is needed can be coupled to the RBC. Or, a non-peptide, non-protein antigen can be linked to a peptide or protein and then the peptide or protein portion of the compound can then be linked to the RBC.

In one embodiment, two or more different peptides/proteins are conjugated to the same RBC. In another embodiment, two or more peptides/proteins that form a complex are conjugated to the RBC using NHS/sulfo-NHS. The complex formed by two or more proteins can have antigens that are not present on the individual proteins.

In still another embodiment, an antigen is coupled to a nonantigenic peptide/protein, and the nonantigenic peptide/protein is then conjugated to the RBCs using NHS/sulfo-NHS cross-linking chemistry.

The amount of protein conjugated to a RBC is about 50-10,000 peptide/protein molecules per RBC, depending on the desired outcome.

The methods and compositions disclosed herein are significantly different and superior compared to existing technologies. NHS and sulfo-NHS esters have never been used to link proteins onto RBCs. The disclosed NHS or sulfo-NHS “activated” peptides/proteins can be lyophilized and stored for convenient on-demand coupling to RBCs, which has not previously been described. Additionally, the activated NHS or sulfo-NHS peptides/proteins may be stored frozen in solution for on-demand coupling to RBCs, which also has not been previously described. Furthermore, the activated NHS or sulfo-NHS peptides/proteins can be lyophilized in a pre-weighed format, such that the user/handler does not have to weigh out the desired quantity of chemical. Having a lyophilized NHS or sulfo-NHS activated peptide/protein allows for the ability to rapidly and conveniently couple peptides/proteins to RBCs. In particular, autologous RBCs from a patient could be rapidly and conveniently coupled to lyophilized NHS or sulfo-NHS activated peptides/proteins and re-infused into an individual in a short time-span (less than 2 hours). Moreover, activated NHS or sulfo-NHS peptides/proteins have never been used to create peptide/protein linked RBCs for immune tolerance induction, immunization, antibody adsorption and elimination, or delivery of compounds to macrophages/antigen-presenting cells. Additionally, intentionally damaging (using heat or chemical agents) peptide/protein linked RBCs to regulate the half-life of the peptide/protein linked RBCs has never been described.

Patients have a small-volume (˜50-100 mL) blood draw performed (˜15 minutes), have their RBCs washed (10-30 minutes), have their RBCs reacted with NHS/sulfo-NHS activated peptides/proteins (10-30 minutes), have their RBCs washed again (10-30 minutes), and then receive a transfusion (15 minutes) of a small-volume (˜50-100 mL) peptide or protein-coupled-RBCs in one visit. The entire process from blood collection to the completion of transfusion could be completed in approximately 2 hours or less. Otherwise, an aqueous solution of activated NHS or sulfo-NHS protein may be administered intravenously for in vivo peptide/protein to RBC coupling. Without lyophilization or freezing of the NHS or sulfo-NHS activated protein esters, the activated protein must be prepared prior to each RBC coupling procedure—requiring significant time and effort which would not be rapid or convenient. This is because NHS or sulfo-NHS protein esters have short half-lives in aqueous or water-containing solutions (approximately 1-5 hours) due to rapid hydrolysis (Hermanson, G T. Bioconjugate Techniques. Academic Press (2013)).

Existing methods to covalently link proteins to RBCs prior to this disclosure are problematic because they either damage the RBCs, are labor-intensive, or are time-consuming (see section “Background of the Invention”).

Additionally, the disclosed compositions and methods are a significant improvement over methods such as internal loading of proteins into RBCs. One well known method of loading proteins into RBCs is technology using hypotonic/hypertonic stress applied to allogenic RBCs. The protein encapsulation process requires 3-8 hours. In addition, the time required to find a compatible donor unit of blood, processing, and shipping requires approximately 24 hours. The internal loading method requires significant time, and large-scale manufacturing processes use allogeneic rather than autologous RBCs. This is unlike the method of using NHS and sulfo-NHS activated peptides/proteins for surface loading onto RBCs. The entire process of collecting blood, washing blood, coupling the peptide/protein, re-washing the blood, and transfusing small volumes of peptide/protein linked RBCs takes less than 2 hours for autologous or allogeneic RBCs.

Strictly defined, there are several key differences between peptides and proteins: peptides are small (100 amino acids or less) while proteins are large (101 amino acids or more). Peptides often require organic solvents in order to be dissolved, while proteins can be dissolved in aqueous solutions. Peptides are often small fragments derived from proteins. However, some full-length compounds (e.g. insulin) are peptides. While peptides have been studied for immune tolerance induction in clinical trials, immune tolerance induction protocols often require the use of full-length proteins (C. Hay, Blood 119(6):1335-44 (2012)). This is because therapeutically infusing a protein for immune tolerance induction allows the human body to process the protein into all possible peptide epitopes which are needed for immune tolerance. However, therapeutically infusing a peptide derived from a protein allows for processing of the peptide into only a limited number of peptide epitopes, which may not be able to induce immune tolerance. Simply put, in order to achieve immune tolerance induction in a clinical setting, proteins rather than peptides are often administered to the patient. Other key differences between proteins and peptides include that proteins have complex 3-dimensional shapes and can perform functions such as enzyme activities, binding to receptors, regulating processes, etc. Peptides fragments from proteins cannot perform these functions. Therefore, it is significant that proteins can now be covalently linked to the surface of RBCs.

B. Red Blood Cells

The RBCs, or erythrocytes, are in blood vessels and transport O₂ bound to hemoglobin. A RBC is flexible and assumes a bell shape as it passes through extremely small blood vessels. The shape of RBCs increases the surface area for the diffusion of oxygen across their surfaces. An RBC is covered with a membrane composed of lipids and proteins, lacks a nucleus, and contains hemoglobin, a red, iron-rich protein that binds oxygen.

With a diameter of only 6-8 μm, RBCs are small enough to squeeze through the smallest blood vessels. Healthy human adult RBCs circulate around the body for up to 120 days, at which point the old or damaged RBCs are removed from the circulation by specialized cells (macrophages/antigen presenting cells) in the spleen, liver, and bone marrow.

RBCs suitable for conjugating to whole proteins are typically autologous, human RBCs obtained from whole blood. Methods for isolating RBCs from whole blood are well known. One method for isolating RBCs is by using differential centrifugation to fractionate blood into RBCs, buffy coat (leukocytes), and plasma. RBCs may be collected using an FDA approved blood collection bag with a blood bag containing an anticoagulant/preservative. Alternatively, small volumes of blood may be collected into a heparinized or otherwise anticoagulated syringe.

### III. Methods of Making Peptide/Protein Conjugated Red Blood Cells

One embodiment provides RBCs conjugated to protein antigens. To conjugate proteins to RBCs, the proteins must be chemically modified. One embodiment provides forming amine-reactive esters from the carboxylic acids of the proteins C-terminus and R-groups of amino acids. Amino acids that have carboxylic acid R-groups include aspartic acid and glutamic acid. The chemistry for forming reactive esters from carboxylic acids is well known (Hermanson, G T. Bioconjugate Techniques. Academic Press (2013)).

One embodiment uses N-hydroxysuccinimide (NHS) with DCC or N-hydroxysulfosuccinimide (sulfo-NHS) with EDC to create dry-stable (amine-reactive) activated protein intermediates. All reactions may be carried out at room temperature or can be carried out at 1-6° C. or 1-10° C., if needed. The peptide/protein of interest can be amine-blocked prior to activation with NHS or sulfo-NHS. The choice to use either NHS or sulfo-NHS is made based on the hydrophobic or hydrophilic property of the peptide/protein. If the peptide/protein is hydrophilic, then sulfo-NHS activation can be easily performed. Sulfo-NHS activation is performed in aqueous solutions (e.g. PBS). Most proteins are hydrophilic, however many peptides are hydrophobic. If the peptide/protein is hydrophobic, then several options exist for activation.

One option is to dissolve the hydrophobic peptide/protein into a small volume of organic solvent (e.g. DMSO) and then mix it with an aqueous solution. Then sulfo-NHS activation can be performed. A second option is that the peptide/protein sequence can be modified to make the peptide/protein more hydrophilic. By the addition or exclusion of amino acids from the N-terminus and/or C-terminus, the peptide/protein can be given a net positive or net negative charge, which will make it more hydrophilic. For best results, the addition and/or exclusion of amino acids should be done such that the amino acid sequence still represents a native sequence of amino acids found in the native peptide/protein. However, the amino acid sequence which is added does not need to be found in the native peptide/protein. If the peptide/protein can then be dissolved in an aqueous solution, then sulfo-NHS activation can be performed. A third option is that NHS activation can be performed in an organic solvent (e.g. dimethyl sulfoxide or DMSO, dimethylformamide or DMF, acetone, dioxane, etc.).

The peptide/protein activation reaction with EDC and sulfo-NHS is most efficient at pH 4.5-7.2. For best results, the peptide/protein should be between 1-10 mg/mL. If denaturation or precipitation of the peptide/protein is a problem, then then pH can be increased to 6.0-7.2. The quantity of EDC and sulfo-NHS should be based on the specific peptide/protein. If there is 1.0 mL of the protein (which is based on a 50 kDa protein) at 10 mg/mL, then add 0.4 mg (2 mM) of EDC and 1.1 mg (5 mM) of sulfo-NHS. For best results, the reaction should be performed in 0.1M MES, 0.9% (0.5 M) NaCl buffer at pH 4.7-6.0. The reaction should be for 10-25 minutes. For best results, the sulfo-NHS activated peptide/protein can be purified from the reaction byproducts by use of buffer-exchange with a desalting column. The desalting column should have an appropriate molecular weight-cut off, such that the peptide/protein passes through the column, but the reaction byproducts (which are less than 1000 daltons) are trapped in the column. An equivalent solution of MES buffer at pH 4.7-6.0 can be used to purify the peptide/protein into MES buffer at pH 4.7-6.0. If necessary, the activated peptide/protein can be purified into a buffer with a pH of 7.2-74. Then, the protein can be frozen and then lyophilized or stored frozen as a solution. NHS/sulfo-NHS activated peptides/proteins have a short half-life in aqueous solution due to hydrolysis of the ester. The half-life is very short when the pH is neutral or basic (Hermanson, G T. Bioconjugate Techniques. Academic Press (2013)). Purifying the sulfo-NHS activated peptide/protein into a buffer with an acidic pH will minimize hydrolysis of the activated peptide/protein. An alternative to using a buffer-exchange spin column, is to use a desalting and gel filtration chromatography.

Amine-blockers such as sulfo-NHS-acetate or other compounds can be reacted with the peptide/protein of interest prior to its “activation” with NHS or sulfo-NHS to block primary amine groups on the protein. By blocking amine groups, intra- and inter-molecular cross-linking of the peptide/protein of interest will not be able to occur. The activated peptide/protein will only be able to cross-link when mixed with RBCs. For best results, the peptide/protein can be at 1-10 mg/dL in PBS. Amine blocking with sulfo-NHS-acetate can be performed by adding a 25-fold molar excess of sulfo-NHS-acetate to the peptide/protein. Sulfo NETS-acetate reacts with primary amines found on the protein of interest. For best results, the reaction should occur at room temperature, for 1-3 hours, in a physiologic buffer which lacks primary amine groups, at a pH of approximately 7.4. The amine-blocked peptide/protein can then be purified using a desalting column. The desalting column can also be used to suspend the amine-blocked peptide/protein in the desired buffer at the desired pH. Or, the amine blocked peptide/protein can be frozen and then lyophilized. NHS and sulfo-NHS can then be used to activate the peptide/protein. Example 1 describes how a protein can be amine blocked and then activated by sulfo-NHS. Example 2 provides information about creating NHS activated peptides/proteins.

To make using NHS/sulfo-NHS activated peptides/proteins easy, the newly synthesized organic or aqueous solution of NHS peptide/protein or sulfo-NHS peptide/protein can lyophilized in known quantities. To do so, the solution of NHS/sulfo-NHS activated peptide/protein should be aliquoted such that a defined quantity of activated peptide/protein is in the solution. Then the solution should be quickly frozen and then lyophilized. This will yield a single-use container with a defined weight of dry NHS/sulfo-NHS peptides/proteins. As an example, 5 mg aliquots of dry activated peptide/protein may be provided in single-use containers. This is preferred to using a single “stock” container which must be re-opened multiple times to measure specified quantities of activated peptide/protein when needed.

RBCs are typically isolated from the blood of the subject to be treated. When blood is drawn from the subject, it is preferably collected in an anticoagulant with or without a preservative. Separating the RBCs from non-RBC components which contain primary amine groups improves the efficiency of linking proteins to the RBCs rather than cross-linking to blood proteins. The RBCs are easily separated from other blood components by centrifugation at the appropriate gravitational force and time settings validated for the centrifuge and blood collection container. Once the RBCs are separated or enriched, the RBCs are washed with a physiologic solution (e.g. PBS) to remove residual plasma, platelets, white blood cells, and blood proteins such as albumin. The final wash should evenly suspend the RBCs in a physiologic solution with a pH of approximately 7.4, which lacks primary amines.

In a biosafety cabinet using sterile or semi-sterile conditions, the washed RBCs are mixed with the NHS or sulfo-NHS peptide/protein with RBCs. The reaction can occur at room temperature. The optimal ratios and concentrations of RBCs and NETS-protein or sulfo-NHS-protein should be determined for the specific protein, buffer, etc. An exemplary ratio of NETS/sulfo-NHS peptide/protein to RBCs is 5-40 mg of activated peptide/protein per 1×10̂9 RBCs in 3-15 mLs of solution. The quantity of the protein which is linked to the RBCs correlates with reaction time (also known as the coupling time). The longer the reaction time, the more peptide/protein which is coupled to the RBC, and vice versa. In general, the first 15 minutes of a 2 hour coupling procedure is when ˜80% of the peptide/protein attaches. Therefore, the quantity of peptide/protein linked to RBCs can be regulated by the reaction time. In addition, the quantity of peptide/protein coupled to the RBC directly correlates with the quantity of activated peptide/protein. Therefore, the quantity of peptide/protein linked to RBCs can be regulated by the quantity of activated peptide/protein reacted with the RBCs. The less protein on the surface of the RBC, the longer the protein-coupled-RBC half-life, and vice versa. The coupling reaction can be stopped after the desired duration by washing the RBCs and removing the supernatant. The peptide or protein coupled-RBCs can then be washed as many times as needed to eliminate any undesired reaction byproducts. The peptide or protein linked RBCs can be suspended to a final hematocrit 25-70%. The peptide or protein linked RBCs are then transfused.

### IV. Methods of Using Protein-Coupled RBCs

A. Promoting Immune Tolerance

Another embodiment provides using peptide/protein coupled RBCs to promote antigen-specific immune tolerance, in which the proteins coupled to the RBC represent the antigen(s) for which tolerance is needed. The RBC can act as a delivery vehicle to target the proteins to antigen-presenting cells of the spleen, liver, and bone marrow for tolerance induction.

Antigen-specific immune tolerance is a highly desired therapy for immune-mediated diseases (Getts, D. R., et al., J. Immunol., 187(5):2405-2417 (2011)). Intravenous infusion of protein or peptide antigens linked to lymphocytes with ethylene carbodiimide (EDC) is a highly efficient method for inducing peripheral, antigen-specific T cell tolerance for the treatment of autoimmune disease. Lutterotti, A. et al. disclose using EDC to couple myelin peptides to autologous peripheral blood mononuclear cells (PBMCs) for treating multiple sclerosis (Lutterotti, A., et al., Sci. Transl. Med. 5, 188ra77 (2013)). However, lymphocytes must be collected in human patients using apheresis, which is expensive and time-consuming. In contrast, RBCs (RBCs) can easily be obtained from a subject using a simple and routine blood draw.

Attempts to induce immune tolerance to peptide antigens by coupling these molecules to RBCs (instead of lymphocytes) are effective. In one experiment, RBCs were coupled to insulin using EDC. These insulin-coupled-RBCs were effective at inducing immune tolerance to type I diabetes (S. Prasad, J Autoimmun. 39(4):347-353 (2012)). However, the insulin-RBCs had to be sonicated before infusion into mice. Otherwise infusion of non-sonicated insulin-coupled-RBCs resulted in immediate hemolysis and toxicity. Kontos, et al. created an ovalbumin antigen linked to a peptide ERY1, which attaches to glycophorin-A found on mouse RBCs. The ovalbumin-linked RBCs were capable of inducing T-cell deletion and immune tolerance. However, this peptide-to-RBC attachment strategy will not work in all humans. Humans have diverse genetics: some have glycophorin A on RBCs, some lack glycophorin A on RBCs, and some have variations in the molecular structure of glycophorin A. Therefore, using a targeting binding strategy to attach protein cargo onto RBCs only works if an antigen is present on the RBCs. Using activated NHS and sulfo-NHS esters to attach proteins to RBCs does not require that a specific molecule be present on the RBC surface. Therefore, using activated NHS and sulfo-NHS esters is a universal method for attaching proteins to RBCs (FIG. 7).

In one embodiment, the antigen is a protein antigen coupled to the primary amines on the surface of the RBCs. In another embodiment, the antigen is a non-protein antigen coupled to a protein, and the antigen bearing protein is coupled to the surface of the RBCs.

Suitable antigens are antigens that produce antibodies in a human subject. Representative proteins that contain antigens to which antibodies are known to be produced include, but are not limited to Factor VIII, asparaginase, myelin, actin, cyclic citrullinated peptide, thrombin, gluten, nuclear proteins, ribonucleoprotiens, snRNP core proteins, histone proteins, nucleoporin 62, Sp100 nuclear antigen, IgG, mitochondrial proteins, nicotinic acetylcholine receptor, muscle specific kinase, nucleoporin 210 kDa, glutamate decarboxylase, glutamate receptor, centromere protein, GAD65, Islet antigen-2 (IA-2), insulin, thyroid peroxidase, thyroglobulin, gliadin, reticulin, 21-hydroxylase, 17-hydroxylase, the cytochrome P450 side chain cleavage enzyme, gastric H+/K+-ATPase, intrinsic factor, Fc portion of IgG, voltage-gated potassium channel, topoisomerase I, ribosomal P, aquaporin-4, N-methyl-D-aspartate receptor, and tRNA synthetase.

Another embodiment provides a method for inducing immune tolerance to an antigen in a subject by administering to the subject autologous or allogeneic RBCs, wherein the antigen is coupled to the surface of the RBCs.

FIG. 9/10 depict how protein linked RBCs can be used to achieve humoral immune tolerance. FIG. 11 depicts how protein-linked RBCs can be used to achieve cellular immune tolerance.

Another embodiment provides a method for treating an immune condition by administering to a subject in need thereof an effective amount of autologous or allogenic RBCs having antigen causing the disease, conjugated to the surface of the RBCs to induce immune tolerance to the antigen in the subject. The number of molecules of peptide/protein per RBC, the number of RBCs, and the half-life of the RBCs needs to be determined for each antigen. For example, 1×10̂9 RBCs with 200 antigen molecules per RBC, which are not heat-damaged, can be used as a starting point. The humoral and cellular immune responses should be studied monthly. If there is no immune tolerance after one month, increase the number of RBCs transfused by a factor of ten. Then, reassess the humoral and cellular immune responses after 1 month. If there is no immune tolerance after the month, increase either the number of RBCs transfused by a factor of ten or increase the number of antigens per RBC by a factor of two-three. Example 3 describes a method for inducing immune tolerance.

Representative immune conditions are that can be treated include, but are not limited to allergies (e.g. peanut allergens), anti-drug antibodies (e.g FVIII, asparaginase), antibodies against tissue/organ transplants (e.g. RBCs, kidney transplants, liver transplants, islets transplants), myasthenia gravis, multiple sclerosis, asthma, celiac disease, systemic lupus erythematosus, rheumatoid arthritis, type 1 diabetes, autoimmune polyendocrine syndrome type 1 (APS-1), and immunodysregulation polyendocrinopathy enteropathy X-linked syndrome (IPEX), and potentially contribute to asthma, allergy, and inflammatory bowel disease.

The disclosed RBC compositions can be administered with a second therapy including, but not limited to immunosuppressive agents. Exemplary immunosuppressive agents include, but are not limited to glucocorticoids, cytostatics, methotrexate, and cytokines.

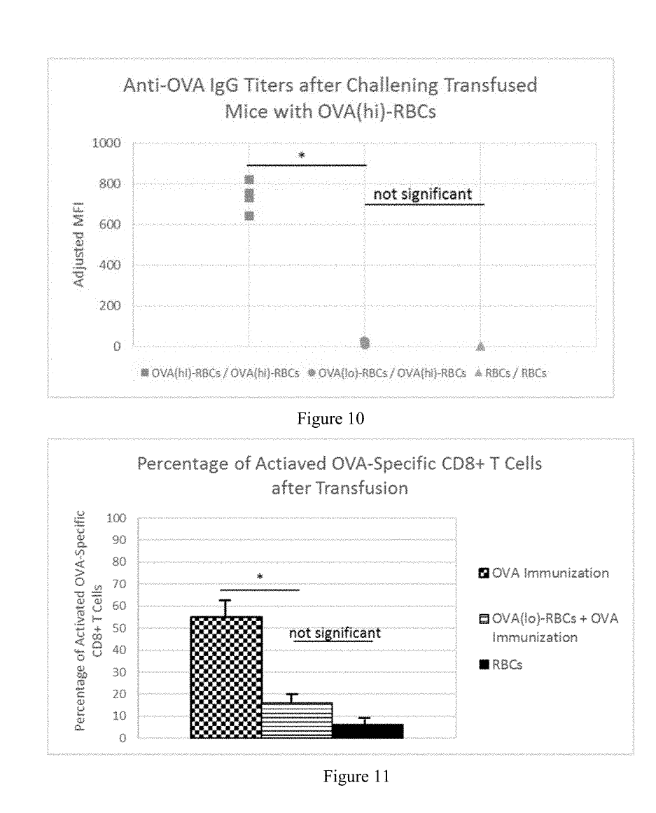

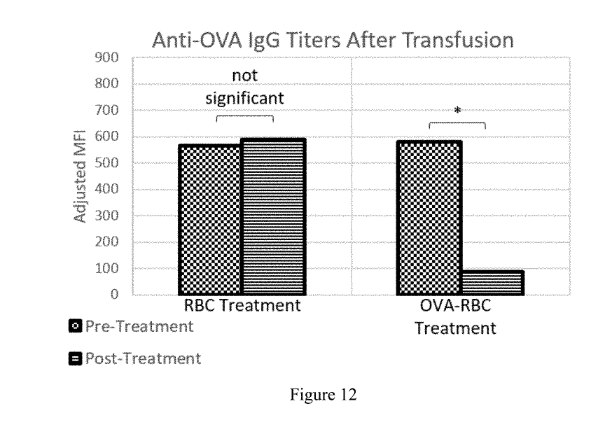

B. Promoting Immunization
Current methods for immunization are problematic for some antigens. By linking the peptide/protein, for which for immunization is needed, onto the RBC, immunization can be achieved. In general, higher numbers of peptide/protein molecules per RBC should be used (FIG. 10). In addition, transfusing both heat-damaged (48.5° C. for 20 minutes) and non-damaged populations of peptide/protein linked RBCs, in a ratio of 1:1 can enhance immunization. An adjuvant (e.g. alum) may also be administered with the RBCs to enhance immunization. If immunization is not achieved, then increasing the number of peptide/protein linked RBCs transfused, increasing the number of peptides/proteins per RBC, and increasing the adjuvant dose should be attempted. Example 4 describes a method for immunization.

C. Removing Pathologic Antibodies

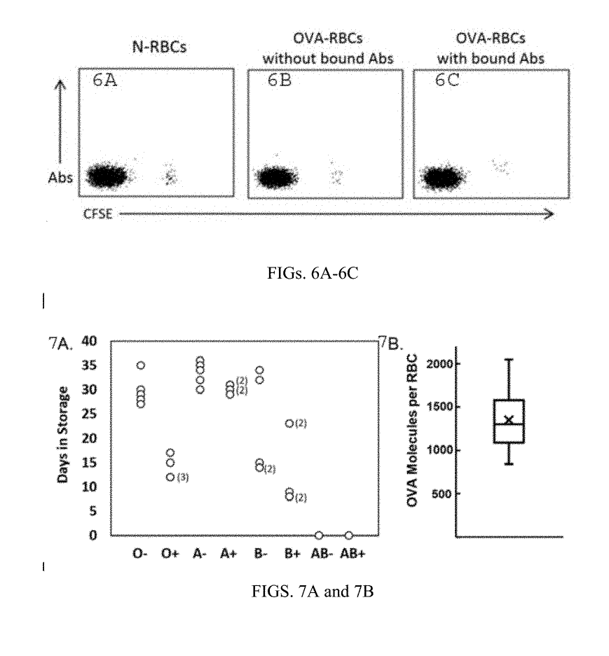

Another embodiment provides using peptide/protein linked RBCs to adsorb and eliminate pathologic antibodies directed against or cross-reactive with the peptide/protein coupled to the RBC. Pathologic antibodies in the blood will bind to the peptide/protein antigen on the RBC (FIG. 6). Then, the antibody-coated-RBCs are eliminated by the macrophages/antigen-presenting cells of the spleen, liver, and bone marrow. Therefore, the antibody levels/titers directed against the peptide/protein will decrease. The antibody levels/titers will decrease over the course of several hours. In addition, peptide/protein linked RBCs can be transfused when antibody titers are low, in order to keep antibody titers low. When peptide/protein linked RBCs bind a threshold amount of antibody, they are cleared from the circulation by the macrophages/antigen-presenting cells of the spleen, liver, and bone marrow. So, if a small quantity of antibody binds to the peptide/protein RBC, the cell will still circulate. It will only be removed when a large (threshold) amount of antibody is attached. If an extremely large quantity of peptide/protein is attached to the RBC, then it is possible that antibody binding could trigger intravascular hemolysis, which is dangerous and undesirable. Therefore, attaching a small quantity of peptide/protein (<1,000 molecules), may be preferred. However, the risk of hemolysis is based not only on quantity of peptide/protein on the RBC, but also properties of the antibody (class, subclass, titer, etc.). Therefore, the parameters should be optimized for the specific antigen. If only a small quantity of peptide/protein is attached to the RBCs, then the RBCs may be able to circulate for a near-normal lifespan. The quantity of peptide/protein attached to RBCs can be adjusted by controlling the incubation time in which the activated peptide/protein is reacted with the RBC. For best results, the amount of activated peptide/protein can be increased or decreased to increase or decrease the amount of peptide/protein attached to the RBC. Because the peptide/protein linked RBCs can circulate for more than 4-weeks in mice, these cells have the ability to adsorb/eliminate pathologic antibodies over the course of weeks after transfusion. If the antibody targets a non-protein antigen such as a lipid, carbohydrate, or nucleic acid, then the lipid, carbohydrate, or nucleic acid can be covalently linked with a peptide/protein (Hermanson, G T. Bioconjugate Techniques. Academic Press (2013)). The peptide/protein can then be activated with NHS/sulfo-NHS and then reacted with the RBC. Example 5 describes a method for specific antibody titer reduction.

D. Regulating Half-Life of the Peptide/Protein Linked to the RBCs, by Damaging the RBCs

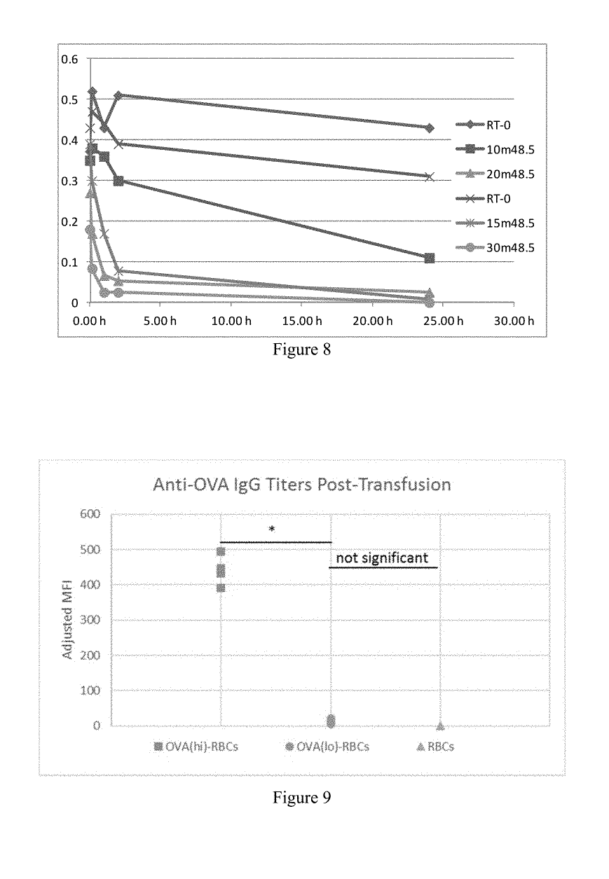

Another embodiment provides using RBCs to control the half-life of the attached peptide/protein. By linking peptides/proteins to RBCs, the half-life of the peptide/protein can be increased or decreased. By attaching the peptide/protein onto fresh, intact, undamaged RBCs, the lifespan of the peptide/protein can be extended beyond 4 weeks in mice. By damaging peptide/protein linked mouse RBCs with heat (48.5° C. in a water bath), the lifespan of the peptide/protein on the RBCs can be decreased (FIG. 8). Because human RBCs are larger than mouse RBCs, thermal damage of human RBCs will have different parameters than for mouse RBCs. Thermal damage should be performed on peptide/protein linked RBCs contained in a blood bag, in a water bath with a regulated temperature setting. Temperatures of approximately 49.0° C. should be used to produce peptide/protein linked RBCs which are cleared from the circulation in approximately 24 hours. In addition, the time of temperature exposure can be controlled, and should be approximately 20 minutes. Example 6 describes a method for regulating the half-life of peptide/protein linked RBCs using thermal damage

Because the RBCs are cleared by macrophages/antigen-presenting cells of the liver, spleen, and bone-marrow, the RBCs can be used to rapidly deliver the peptide/protein cargo to these cells. Another therapeutic use of protein-coupled-RBCs is to deliver drugs (attached to the peptide/protein) to macrophages/antigen-presenting cells in the spleen, liver, and bone marrow. The uses of protein-coupled-RBCs are not limited to those stated herein.

### VI. Kits

Kits for preparing RBCs conjugated with peptides/proteins are also provided. The kits include a container housing aliquots of lyophilized Sulfo-NHS-activated peptide(s)/protein(s) or NHS-activated peptide(s)/protein(s). The kit also includes pH 7-8 buffered solution for reacting the activated protein(s) with RBCs. PBS (100 mM sodium phosphate, 0.15M NaCl; pH 7.4) is a preferred solution. In addition, an organic solvent, such as DMSO, may be included if the peptide/protein needs to be initially dissolved in an organic solvent before it can be dissolved in an aqueous solvent. The kit may also include a device/equipment for easily isolating RBCs from whole blood and for washing RBCs. Such microfluidics devices/equipment are currently being developed for blood processing. In addition, the kit may also include reagents/testing supplies for confirming that the peptide/protein is attached to the RBC. For example, an antibody with specificity against the peptide/protein may be provided. This primary antibody may be fluorescently labelled. Or, a secondary antibody may be provided, which is fluorescently labelled, with specificity for the primary antibody. The antibodies can be used in conjunction with platforms such as flow cytometry to confirm the RBCs are linked with peptides/proteins. If the RBCs are linked with peptides/proteins, then the RBCs will appear fluorescent using flow cytometry. Additionally, a card or cassette with bound antibodies targeting the peptide/protein attached the RBCs may be included in the kit, to rapidly confirm the attachment of the peptide/protein onto the RBC. The kit also includes instructions for coupling the activated peptide/protein antigens to the RBCs.

### EXAMPLES

### Example 1

### Coupling Proteins to Red Blood Cells

The following is an exemplary protocol for preparing protein conjugated RBCs. The protein ovalbumin made by Gallus gallus (UniProt P01012) has a molecular weight of more than 42,000 Daltons. For best results, use sulfo-NHS-acetate (1.23 mg/6 uL PBS buffer pH 7.2-7.6 without primary amines) to block primary amine groups on ovalbumin (0.39 mg/39 uL PBS ph 7.2-7.6) for 0.5-3 hours at room temperature. The reaction should occur in a non-amine containing aqueous solution (such as PBS at pH of 7.2-7.6). Purify the amine-blocked-ovalbumin using a desalting column with a buffer exchange protocol (spun at 1000 g-1500 g for 2 minutes) to purify the protein into a non-carboxylate and non-amine buffer (such as 2-(N-morpholino) ethanesulfonic acid, also known as MES buffer) at pH 5-6. Amine-blocking is not necessary and the steps for amine blocking can be omitted if it is not needed. Mix the amine-blocked or non-amine-blocked ovalbumin (approximately 0.39 mg in approximately 39 uL of buffer) with sulfo-NHS (0.82 mg/4 uL MES buffer pH 5-6) and EDC (0.3 mg/4 uL MES buffer pH 5-6) and let the reaction proceed for 10-20 minutes. Purify the sulfo-NHS activated ovalbumin using a desalting column with a buffer exchange protocol (spun at 1500 g for 2 minute) to elute the protein in an amine-free buffer such as PBS at pH 7.4. Either react the sulfo-NHS activated ovalbumin with washed RBCs or immediately freeze at −2 or −80° C. If the sulfo-NHS activated ovalbumin is frozen, then it can be lyophilized and stored as a dry chemical. Obtain a whole blood sample in an anticoagulant (e.g. heparin, EDTA, citrate). Isolate the RBCs from the plasma, platelets, and white blood cells by centrifugation (100-6000 g) for the appropriate length of time for the volume of blood collected. Wash RBCs using a manual procedure or automated cell washer. Washing using a centrifuge involves spinning the blood to obtain a bottom fraction of RBCs and a top fraction of fluid. If performing a manual wash with a centrifuge, perform 1-4 wash steps with an amine-free buffer at pH 7.4. Alternatively, RBCs can be washed with newer washing devices which use microfluidics. Mix the sulfo-NHS “activated” ovalbumin with 1×10⁸ RBCs. Gentle agitation or mixing can be performed using a device such as a rocking platform. Let the reaction proceed for the desired length of time (10 minutes—2 hours) at room temperature. Wash the ovalbumin-coupled-RBCs with PBS at pH 7.4, 1-3 times to remove undesired reaction byproducts. The hematocrit of the peptide or protein linked RBCs can be suspended to a final hematocrit 25-70%. The ovalbumin-coupled-RBCs are ready for transfusion. Of note, the coupling procedure may vary depending on the characteristics of the peptide/protein.

Table 1 contains a comparision of methods used to link proteins to RBCs.

### Example 2

### Activating Peptides with NHS

The following is an exemplary protocol for activating peptides which are not soluble in aqueous solution. Dissolve 3.45 mg of NHS into 30 mL of dry ethyl acetate. Add ˜20 mMol of peptide to the NHS solution. Dissolve 6.18 gm of DCC into 10 mL of ethyl acetate and add it to the solution. Let the mixture react at room temperature overnight. For best results, perform the reaction with a nitrogen atmosphere over the solution. An insoluble precipitate, known as dicyclohexyl urea (DCU) can be removed using filtration (e.g. glass-fiber filter pad with vacuum). Then, remove the solvent from the filtered solution using a rotary evaporator under a vacuum. The NHS activated peptide can then be purified. Dissolve the NHS activated peptide into a small volume of hot ethanol. Filter the solution through a filter funnel containing a fluted glass-fiber filter pad which has been warmed to the temperature of the ethanol solution. Allow overnight recrystallization. Remove solvent by filtration. Then, dry using a vacuum in a desiccator.

### Example 3

### Inducing Antigen-Specific Immune Tolerance with Peptide/Protein Linked RBCs.

Select the peptide/protein antigen to which immune tolerance is needed. Create peptide/protein linked mouse RBCs with ˜200 peptide/protein molecules per RBC. Wash away reaction byproducts. Transfuse the peptide/protein linked mouse RBCs into mice within 4 hours of creation. Wait at least 2 weeks. Evaluate the antigen-specific cellular and/or humoral immune responses. The results should demonstrate immune tolerance. Otherwise, the number of RBCs, the number of peptide/protein molecules per RBC, or the half-life of the RBCs may need to be modified.

### Example 4

### Immunizing with Peptide/Protein Linked RBCs

Select the peptide/protein antigen for which immunization is needed. Create peptide/protein linked mouse RBCs with ˜1000 peptide/protein molecules per RBC. Wash away reaction byproducts. Heat damaged half of the peptide/protein linked RBCs. Transfuse both the heat damaged and non damaged peptide/protein linked mouse RBCs into mice within 4 hours of creation. Wait at least 3 weeks. Evaluate the specific antibody titers. The results should demonstrate an increase of specific antibody titers.

### Example 5

### Reducing Specific Antibody Titers using Peptide/Protein Linked RBCs.

Select the peptide/protein antigen which binds the specific antibody for which the titer should be reduced. Create peptide/protein linked mouse RBCs with ˜1000 peptide/protein molecules per RBC. Wash away reaction byproducts. Transfuse the peptide/protein linked mouse RBCs into mice within 4 hours of creation. Wait at least 2 hours. Evaluate the specific antibody titers. The results should demonstrate reduction of specific antibody titers.

### Example 6

### Regulating the Half-Life of Peptide/Protein Linked RBCs.

Peptide or protein linked RBCs, which are freshly made, are suspended at a hematocrit of 25-60%. The RBCs should be in a blood bag, and then placed in a secondary spill bag. The RBCs should then be submerged into a water bath which is set to approximately 48.5° C. The RBCs should be exposed to the water bath for a desired amount of time. Fifteen minutes of heating will damage the

RBCs so that they will be rapidly cleared from the circulation (within 48 hours) after circulation. The RBCs are then cooled by being placed at 1-10° C. for 20 minutes. The RBCs should be released for transfusion within 1 hour after cooling.

While in the foregoing specification this invention has been described in relation to certain embodiments thereof, and many details have been put forth for the purpose of illustration, it will be apparent to those skilled in the art that the invention is susceptible to additional embodiments and that certain of the details described herein can be varied considerably without departing from the basic principles of the invention.

All references cited herein are incorporated by reference in their entirety. The present invention may be embodied in other specific forms without departing from the spirit or essential attributes thereof and, accordingly, reference should be made to the appended claims, rather than to the foregoing specification, as indicating the scope of the invention.

We claim:

1. A composition comprising autologous or allogenic red blood cells (RBCs) having peptide(s) or protein(s) convalented conjugated to the surface of the RBCs by activating the peptides or proteins in a reaction with N-hydroxysuccinimide (NHS) or N-hydroxysulfosuccinimde (sulfo-NHS) in the presence of 1-ethyl-3(3-dimethylaminopropyl)carbodiimide (EDC) or dicyclohexylcarbodiimde (DCC).

2. The composition of claim 1, wherein the reaction occurs in either an organic solvent or aqueous non-carboxylic acid containing buffer solution.

3. The composition of claim 2, wherein the peptide(s) or protein(s) are incubated with a blocking agent (sulfo-NHS-acetate), thereby blocking the free amine groups on the peptide/protein.

4. The composition of claim 3, wherein the peptide(s) or protein(s) are modified prior the reaction.

5. The composition of claim 4, wherein the activated peptide or protein in is frozen in solution or lyophilized for storage.

6. A kit comprising:
an aliquot of pre-weighed, activated peptide(s) or protein(s) of claim 1;
a sterile physiologic pH 7.2-7.6 buffer solution;
supplies for washing red blood cells;
reagents/supplies for confirming that the peptide/protein is attached to the red blood cells;
and instructions for conjugating the activated peptide(s)/protein(s) to red blood cells obtained from a subject.

7. A method for promoting immune tolerance to an antigen in a subject in need thereof, comprising:
administering autologous or allogenic RBCs to the subject, wherein the RBCs comprise peptide(s) or protein(s) conjugated to the RBCs, and wherein the peptide or protein acts as an antigen for promoting immune tolerance.

8. A method for clearing an antibody from a subject in need thereof comprising: administering autologous or allogenic RBCs to the subject, wherein the RBCs comprise peptide(s)/protein(s) conjugated to the RBCs, and wherein the peptide/protein displays an antigen or cross-reactive compound to which the antibody is specific and wherein the antibody binds the antigen and is neutralized.

9. A method for modulating the half-life of administered peptide or protein coupled RBCs by increasing or decreasing the quantity of peptide/protein attached per RBC.

10. A method for damaging RBCs to reduce the half-life of RBCs.

11. The method of claim 10, wherein the peptide or protein coupled RBCs are heat-damaged inducing spherocytosis

12. The method of claim 11, wherein, the peptide or protein coupled RBCs are sonicated.

13. The method of claim 8, wherein the protein is coagulation Factor VIII or peptide(s) from coagulation Factor VIII.

14. A method for promoting immune tolerance to the protein Factor VIII in a subject in need thereof comprising:
administering autologous or allogenic RBCs to the subject, wherein the autologous red blood cells are conjugated to the protein coagulation Factor VIII or peptide(s) from coagulation Factor VIII, and wherein the Factor VIII contains an antigen for promoting immune tolerance to Factor VIII.

15. The method of claim 8, wherein the autologous or allogenic red blood cells are administered to the subject over a period of days, weeks or years.

16. A method for treating an immune condition in a subject in need thereof comprising administering to the subject an effective amount of autologous or allogenic RBCs, wherein the antigen causing the immune condition is conjugated to the surface of the RBCs and induces immune tolerance to the antigen.

17. The method of claim 16, wherein the immune condition is an autoimmune disease, an allergen, an anti-drug antibody, an antibody against cellular/tissue/organ transplants, alopecia areata, alloimmunization to platelet/red blood cell antigens, autism and autism spectrum disorders, autoimmune hemolytic anemia, autoimmune polyendocrine syndrome type 1 (APS-1), autoimmune hepatitis, asthma, anti-glomerular basement membrane disease, bullous pemphigoid, celiac disease, Crohn's disease, dermatomyositis, Evans syndrome, Graves's disease, Goodpasture's syndrome, glomerulonephritis, Guillain-Barre syndrome, idiopathic thrombocytopenic purpura, immunodysregulation polyendocrinopathy enteropathy X-linked syndrome (IPEX), inflammatory bowel diseases, Lambert-Eaton syndrome, mixed connective tissue disease, myasthenia gravis, multiple sclerosis, neuromyelitis optica, pediatric autoimmune neuropsychiatric disorders associated with streptococcus (PANDAS), psoriasis, polymyositis, restless legs syndrome, rheumatoid arthritis, systemic lupus erythematosus, Sjogren's syndrome, stiff person syndrome, scleroderma, transverse myelitis, type 1 diabetes, thrombotic thrombocytopenic purpura, and vitiligo.

18. A method for eliminating pathologic antibodies comprising infusing/transfusing into a patient with pathologic antibodies a therapeutically effective amount of peptide or protein coupled RBCs comprising an antigen recognized or cross-reaction with the pathologic antibodies, and wherein the pathologic antibodies specifically bind to the peptide or protein coupled to the RBCs, and the antibody-coated RBCs are eliminated by antigen-presenting cells of the spleen and liver.

19. A method for immunizing a subject in need thereof, comprising administering to the subject an effective amount of autologous or allogeneic RBCs, wherein the antigen to be immunized is conjugated to the surface of the RBCs and induces immunization to the antigen.

20. A method of adding and/or removing amino acids to the peptide/protein sequence to create a water soluble peptide/protein which can be activated using sulfo-NHS.
